(19) United States
(12) Patent Application Publication
(10) Pub. No.: US 2021/0144937 A1
(43) Pub. Date: May 20, 2021

(54) BIODEGRADABLE SUBSTRATE FOR SUPPORTING PLANT GROWTH

(71) Applicant: BIOCOMPOSITES GROUP INC., Drayton Valley (CA)

(72) Inventors: Mike Steve Szeman, Alberta (CA); Daniel William Madlung, Alberta (CA)

(73) Assignee: BIOCOMPOSITES GROUP INC., Drayton Valley, AB (CA)

(21) Appl. No.: 16/642,569

(22) PCT Filed: Sep. 6, 2019
(86) PCT No.: PCT/CA2019/051247

§ 371 Date: Feb. 27, 2020

Related U.S. Application Data
(60) Provisional application No. 62/731,420, filed on Sep. 14, 2018.

(51) Int. Cl. A01G 24/22; C05F 5/00; C05G 5/40; A01G 24/35; A01G 24/30; A01G 24/44

(52) U.S. Cl. CPC A01G 24/22; C05F 5/002; A01G 24/44; A01G 24/35; A01G 24/30; C05G 5/40

(57) ABSTRACT

The present invention provides a biodegradable substrate for supporting growth of seeds or clones. The substrate comprises: about 70% to about 95% hemp fibre and about 5% to about 30% of a biodegradable thermoplastic polymer. The present invention also provides a process for preparing a biodegradable substrate for supporting growth of seeds or clones from a hemp fibre-based mat.

### FIELD OF THE INVENTION

The invention relates to substrates for supporting growth of plants and more particularly to biodegradable substrates formed from natural fibres for supporting seeds or clones during germination and subsequent root propagation and emergence of leaves.

### BACKGROUND

Hydroponics is a subset of hydroculture which encompasses methods of growing plants without soil, using mineral nutrient solutions in a water solvent. Terrestrial plants may be grown with only their roots exposed to the mineral solution, or the roots may be supported by an inert medium, such as perlite or gravel. The nutrients in hydroponics can come from an array of different sources.

Initiating growth of a plant from a seed or a clone in hydroponics processes can be challenging. Solutions which have been developed to address this issue include seed-substrate products known as “growing cubes,” and “starter plugs” which provide aeration and moisture retention to enhance germination of the seed and subsequent growth of the roots and other parts of the plant. One prominent example is a material known as ROCKWOOL® which is formed of processed basaltic rock. Peat moss and combinations of wood pulp have also been used, examples of which include Jiffy® seed pellets and Roto Rooter™ plugs.

There remains a need for development of additional substrate products for initiating growth of plants from seeds and clones in hydroponics processes.

### SUMMARY

According to one aspect of the invention, there is provided a substrate for supporting growth of a seed or a clone, the substrate comprising: about 70% to about 95% hemp fibre and about 5% to about 30% of at least one biodegradable thermoplastic polymer.

In some embodiments, the substrate comprises about 75% to about 85% of the hemp fibre and about 15% to about 25% of the at least one biodegradable thermoplastic polymer.

In some embodiments, the substrate comprises about 80% of the hemp fibre and about 20% of the at least one biodegradable thermoplastic polymer.

In some embodiments, the hemp fibre is coated with about 0.3% to about 1.0% (m/m) of a wetting agent. For greater clarity, the percentages are expressed as percentage mass of the wetting agent added to the mass of hemp fibre.

In some embodiments, the wetting agent is a plant extract containing an amphipathic glycoside.

In some embodiments, the plant extract is yucca extract.

In some embodiments of the substrate, the hemp fibre has an average size of about 1 mm to about 10 mm.

In some embodiments of the substrate, the hemp fibre has an average size of about 1 mm to about 50 mm.

In some embodiments of the substrate, the hemp fibre is obtained from whole-stalk processing of hemp plants.

In some embodiments of the substrate, the hemp fibre include bast fibre and hurd fibre.

In some embodiments of the substrate, the biodegradable thermoplastic polymer is in a fibre form.

In some embodiments of the substrate, the biodegradable thermoplastic polymer is compostable.

In some embodiments of the substrate, the biodegradable thermoplastic polymer is polylactic acid, a polyhydroxyalkanoate, a polyesteramide, a polycaprolactone, thermoplastic starch, or poly(butylene adipate co-terephthalate) or a derivative or formulation thereof, wherein the formulation comprises one or more stabilizing additives.

In some embodiments of the substrate, the biodegradable thermoplastic polymer is polylactic acid, or a derivative or formulation thereof, wherein the formulation comprises one or more stabilizing additives.

In some embodiments of the substrate, the substrate has a material basis weight between about 2.0 kg/m² to about 6.5 kg/m².

In some embodiments of the substrate, the substrate has a material basis weight between about 3.5 kg/m² to about 5.0 kg/m².

In some embodiments of the substrate, the substrate has a material basis weight between about 3.5 kg/m² to about 6.0 kg/m²

In some embodiments of the substrate, the substrate has a material basis weight of about 3.5 kg/m².

In some embodiments of the substrate, the hemp fibre and the biodegradable thermoplastic polymer are needle punched to create fibre entanglements therebetween in a fibre air-lay blending process

In some embodiments of the substrate, the substrate is in the form of a block having dimensions of about 30 mm×about 30×about 30 mm to about 40 mm×about 40 mm×about 40 mm and having a hole formed substantially centrally on one side for placement of the seed or the clone.

Another aspect of the invention is a process for forming a hemp fibre substrate in the form of cubes, blocks or slabs for supporting growth of a seed or a clone, the process comprising: a) air-lay blending of hemp fibre and a biodegradable thermoplastic polymer fibre to form a mat; b) heating the mat; c) molding the mat in a molding tool at or below about room temperature to form the mat into a slab; and d) cutting the slab into the cubes, blocks or slabs.

In some embodiments of the process, step a) comprises air-laying of hemp fibre to provide the mat with about 70% to about 95% of the hemp fibre and air-laying of the biodegradable thermoplastic polymer which is then air-lay blended with the hemp fibre to provide the mat with about 5% to about 30% of the biodegradable thermoplastic polymer.

In some embodiments of the process, step a) comprises air-laying of hemp fibre to provide the mat with about 75% to about 85% of the hemp fibre and air-laying of the biodegradable thermoplastic polymer which is then air lay blended with the hemp fibre to provide the mat with about 15% to about 25% of the biodegradable thermoplastic polymer.

In some embodiments of the process, step a) comprises air-laying of hemp fibre to provide the mat with about 80% of the hemp fibre and air-laying of the biodegradable thermoplastic polymer which is then air-lay blended with the hemp fibre to provide the mat with about 20% of the biodegradable thermoplastic polymer.

In some embodiments of the process, the hemp fibre has an average size of about 1 mm to about 10 mm.

In some embodiments of the process, the hemp fibre has an average size of about 1 mm to about 50 mm.

In some embodiments of the process, the hemp fibre is obtained from whole-stalk processing of hemp plants.

In some embodiments of the process, the hemp fibre includes bast fibre and hurd fibre.

In some embodiments of the process, the biodegradable thermoplastic polymer is compostable.

In some embodiments of the process, the biodegradable thermoplastic polymer is polylactic acid, a polyhydroxyalkanoate, a polyesteramide, a polycaprolactone, thermoplastic starch, or poly(butylene adipate co-terephthalate) or a derivative or formulation thereof, wherein the formulation comprises one or more stabilizing additives.

In some embodiments of the process, the biodegradable thermoplastic polymer is polylactic acid, or a derivative or formulation thereof, wherein the formulation comprises one or more stabilizing additives.

In some embodiments of the process, the amounts of the hemp fibre and the biodegradable thermoplastic polymer fibre are adjusted in step a) to provide a mat having a material basis weight between about 2.0 kg/m² to about 6.5 kg/m².

In some embodiments of the process, the amounts of the hemp fibre and the biodegradable thermoplastic polymer fibre are adjusted in step a) to provide a mat having a material basis weight between about 3.5 kg/m² to about 5.0 kg/m².

In some embodiments of the process, the amounts of the hemp fibre and the biodegradable thermoplastic polymer fibre are adjusted in step a) to provide a mat having a material basis weight between about 3.5 kg/m² to about 6.0 kg/m².

In some embodiments of the process, the amounts of the hemp fibre and biodegradable thermoplastic polymer fibre are adjusted in step a) to provide a mat having a material basis weight between of about 3.5 kg/m².

In some embodiments of the process, the molding tool comprises protrusions to form holes in one side of the slab, wherein the holes are provided for placement of the seed or the clone.

In some embodiments of the process, the cubes have dimensions of about 30 mm×about 30×about 30 mm or blocks having dimensions of about 40 mm×about 40 mm×about 40 mm.

In some embodiments of the process, the cubes, blocks or slabs are: plant starters in the form of 0.5″ cubic, 1″×1″×1.5″, 1.5″ cubic, or 2″×2″×1.5″ blocks for use as plant starters; 8″×8″×8″ blocks for growing larger plants and small trees; 3″×3″×2.5″, 3″×3″×4″; 3″×3″×2.5″, 4″×4″×3.1″ or 4″×4″×4″ blocks for transplant of plants with larger roots; 6″×6″×6″ blocks for growing plants up to 3-4 feet tall; 9.5″×8″×4″ slabs for growing herbs and small plants; or 6″×3″×36″, 8″×3″×36″, 6″×4″×36″, 12″×3″×36″, or 8″×4″×9.5″ slabs for growing large vine crops.

In some embodiments of the process, step c) comprises heating to provide a core temperature of the mat of about 150° C. to about 170° C. for between about 30 minutes to about 40 minutes or comprises heating to provide a core temperature of the mat of about 150° C. to about 175° C. for between about 30 minutes to about 60 minutes.

### DETAILED DESCRIPTION

### Rationale and Introduction

With the recent rapid growth of the cannabis industry in North America, large scale processes for initiating plant growth from seeds are being developed and shortcomings regarding the currently favored ROCKWOOL® substrate have been identified. This material is based on processed basaltic rock and disposal or recycling is challenging. In addition, ROCKWOOL® is recognized as a skin irritant. Companies engaged in large scale growth of cannabis plants from seeds are examining all parts of the process and are recognizing that alternatives to ROCKWOOL® which are biodegradable and sustainable would be preferable.

The present inventors are engaged in manufacture of hemp fibre mats for various applications and have recognized that a hemp fibre-based substrate of a particular range of densities and provided with certain properties to enhance aeration and water retention, could provide a useful alternative to ROCKWOOL® and other known seed starter substrate materials. The present application describes compositions and processes for preparing seed starter substrates. The compositions described herein include hemp fibres and biodegradable thermoplastic adhesive polymers, provided to consolidate the hemp fibres and provide a more rigid matrix structure. As described herein, prototype versions of these compositions are capable of supporting seed growth generally equivalent to seed growth supported by ROCKWOOL® and other substrates, while providing the advantage of being biodegradable and formed in majority from portions of the hemp plant which would otherwise be considered waste. This aspect of sustainability is particularly attractive to companies engaged in large scale production of cannabis for recreational and medicinal purposes as it allows a plant growth by-product to be used to support new seed growth.

A number of possible alternative features are introduced during the course of this description. It is to be understood that, according to the knowledge and judgment of persons skilled in the art, such alternative features may be substituted in various combinations to arrive at different embodiments of the present invention.

### Preparation of Substrate Material

As noted hereinabove, seed-supporting substrates are required to provide sufficient aeration and water retention properties to promote germination and growth of seeds. The present inventors have recognized that their expertise in forming hemp mats with specific properties for other applications could be used in preparation of seed supporting substrates such as growth cubes.

It was recognized by the inventors that a mat formed of hemp fibres and a biodegradable thermoplastic polymer providing adhesive properties could provide the desired characteristics for a seed-supporting substrate. A thermoplastic, or thermosoftening plastic, is a plastic polymer, that becomes pliable or moldable above a specific temperature and solidifies upon cooling. The hemp fibres are obtained from bast fibre and hurd (the woody core material of the whole stalk of the hemp plant in a process requiring whole stalk processing). In recognizing that smaller fibres are more effective than long fibres at wicking water, in pre-processing, these fibres are reduced to micro-sized fibres. Tools such as a hammer mill may be used to generate micro-sized fibres.

It was recognized that provision of a wetting agent provides the hemp mat with favorable properties such as reducing the soaking time required for complete saturation, as well as reducing the presence of dry areas. Wetting agents are substances which reduce the surface tension of water to permit water to spread more effectively across a surface. Surfactants such as detergents can function as wetting agents. As it was desirable to use all natural biodegradable components in preparation of the seed-supporting substrate, it was recognized that natural saponins would be useful as wetting agents. Saponins are amphipathic glycosides which produce soap-like foams. Yucca powder was identified as a potentially useful source of amphipathic glycosides. Other natural amphipathic glycosides from other plant sources such as plants of grains, beans, vegetables and herbs, such as soapwort, soaproot, soapbark and soapberry, for example, may be reasonably expected to provide similar properties and advantages.

Polylactic acid was selected as the biodegradable thermoplastic polymer for initial formulations of the hemp-based fibre substrates. The National Zero Waste Council of Canada has confirmed that polylactic acid polymer is both biodegradable and compostable. Therefore, it is particularly advantageous for large scale seed growth operations which include composting processes in efforts to maximize returns from inputs. Characteristics and advantages of polylactic acid fibres have been outlined by Farrington et al. in Biodegradable and Sustainable Fibres, 2005, Woodhead Publishing in Textiles, R. S. Blackburn (Ed) Chapter 6: Poly(lactic acid) Fibers pp 191-220 (incorporated herein by reference in its entirety). It is important to recognize that compostability is not equivalent to biodegradability. Composting is a beneficial waste management system that allows organic materials to be recycled into a product that can be used as a valuable soil additive. For example, the primary mechanism of degradation of polylactic acid is hydrolysis, catalyzed by temperature, followed by bacterial attack on the fragmented residues. In composting, the moisture and the heat in the compost pile attacks the polylactic acid polymer chains and splits them apart, creating smaller polymer fragments, and finally, lactic acid. Microorganisms, found in active compost piles, consume the smaller polymer fragments and lactic acid as an energy source. Since lactic acid is widely found in nature, several naturally occurring organisms metabolize lactic acid. Bacteria and fungi are involved in polylactic acid degradation. The products of the process include carbon dioxide, water and humus, a useful soil additive. In contrast, biodegradability is more general in nature and refers to the ability of a substance to be decomposed by bacteria or other organisms without necessarily producing useful products. Other biodegradable thermoplastic polymers may also be used in alternative embodiments if compostability is not of particular concern, examples of such alternative biodegradable thermoplastic polymers include, but are not limited to, polyhydroxyalkanoates, polyesteramides, polycaprolactones, thermoplastic starches, and poly(butylene adipate co-terephthalate), as well as derivatives or formulations thereof comprising stabilizing additives in minor quantities.

In a first set of experiments described hereinbelow, the biodegradable thermoplastic polymers used in preparing the hemp fibre substrate formulations were in fibre form as proprietary preparations of polylactic acid obtained from two different sources; Pond Global Biomaterials of Aarhus, Denmark (product name: POND BIO FIBER™), and Fiber Innovation Technology Inc. of Johnson City, Tenn., USA (product name: FIT PLA Staple Fiber™). FIT PLA Staple Fiber™ is indicated by the manufacturer to be polylactic acid greater than 99% by weight combined with a proprietary “fiber finish” representing less than 1% by weight of a blend of non-ionic and ionic surfactants and anti-static agents. POND BIO FIBER™ is indicated by the manufacturer to be a polylactic acid derivative generated from biological materials which is as compostable as polylactic acid itself. In both cases, the low levels of proprietary additives or the modified forms of the polylactic acid are not believed to exert any appreciable effect on the desired thermoplastic properties of the polymer in terms of its desired function in consolidation of the hemp fibres in increasing the durability of the hemp fibre mat used as the substrate material.

Mats were constructed in a fibre air-lay blending process with equipment provided for separately laying and blending short fibres and long fibres to create a mat. The process provides an even distribution of fibres in the direction of the process and perpendicular to the direction of the process, to ensure construction of an even, homogeneous mat. The process may include needle punching of the mat formed of the two different fibres. The needle punching causes the fibres to become entangled and this imparts a high tensile strength to the finished mat.

The mat is placed into a convection oven or a forced air oven and heated for about 30 to about 40 minutes or about 30 minutes to about 60 minutes to provide a core mat temperature from about 150° C. to about 170° C. or about 150° C. to about 175° C. to melt the biodegradable thermoplastic polymer to convert it to an adhesive and to kill any spores which may be present. The mat is removed from the oven and immediately molded using a cold molding tool (at or below room temperature) to remove heat from the mat and consolidate and form the mat into a homogeneous slab. The molding tool includes protrusions which form holes in one side of the slab which are used for placement of a seed or clone. The homogeneous slab is then cut into cubes in a manner where the cubes remain held together with some residual fibres so that the cubes can be torn away from each other in a press using a steel-rule die. In one example, a 7″×7″ cube steel-rule die generates a set of 49 hemp fibre substrate blocks are generally cubic and have dimensions of about 35 mm×about 35 mm×about 35 mm. The result is a set of hemp fibre substrate blocks having sufficient tensile strength to retain consistency over the course of the experiments, while retaining sufficient moisture and providing sufficient aeration to promote seed germination and plant growth. Alternatively, a circular saw, blade or other cutting tool can be used instead of using a die to make the cuts.

The material basis weight of the mat is easily controlled in the process and as such, material basis weights ranging from about 300 g/m² to about 6.5 kg/m² can be prepared with thicknesses ranging from about 5 mm to about 100 mm or about 5 mm to about 150 mm. The term “material basis weight”, as used in the pulp and paper and the fabric industries, is the areal density of a paper or fabric product, which can be expressed as mass per unit of area.

### EXAMPLES

### Example 1: Comparison of Hemp Fibre Substrate Blocks with Conventional Growing Cubes

Rationale—Currently there are several options for growers when it comes to choosing a starter substrate for their seeds or clones. Coconut fibre (coir), peat moss, ROCKWOOL®, and combinations of wood pulp are some of the common examples of substrates used in both greenhouse production systems and hydroponic systems. This study explores using hemp fibre substrates in a block form as an alternative seed supporter substrate.

Objectives—The objectives of the study were to (i) evaluate 4 prototypes of hemp-based substrates for greenhouse/hydroponic production; (ii) determine which of the prototypes has the best performance; and (iii) determine if the hemp-based substrates will perform as well as the current industry standards.

Methodology—Four hemp fibre-based substrates were prepared according to processes described hereinabove using two different sources of compostable and biodegradable polymer based on polylactic acid in fibre form which were obtained from Pond Global Biomaterials (Aarhus, Denmark) and from Fiber Innovation Technologies (Johnson City, Tenn., USA). The hemp fibre substrates were formed with two different material basis weights; 3.5 kg/m² and 5.0 kg/m². Shorthand notation for the four substrates refers to the material basis weight and source of the biodegradable polymer; 3.5F is the 3.5 kg/m² material basis weight hemp fibre with FIT PLA Staple Fiber™; 3.5P is the 3.5 kg/m² material basis weight hemp fibre with POND BIO FIBER™; 5.0F is the 5.0 kg/m² material basis weight hemp fibre with FIT PLA Staple Fiber™; and 5.0P is the 5.0 kg/m² material basis weight hemp fibre with POND BIO FIBER™. These four different prototype hemp fibre substrates were compared with conventional non-sustainable materials ROCKWOOL® (basaltic rock heated and spun); Jiffy® seed Pellets (sphagnum peat moss, lime and fertilizer), and Rapid Rooter™ grow plugs (sphagnum peat moss-based sponge material). All four of the hemp fibre substrate formulations included 80% short-length hemp fibres (average length between about 1 mm to about 10 mm) and 20% of the indicated biodegradable thermoplastic polymer.

The four hemp-based prototype substrates and the three conventional substrates were soaked for 30 minutes in reverse-osmosis (RO) water and placed in starter trays in a randomized complete block design with 4 replications on Mar. 20, 2018. Each series consisted of 20 individual starter substrates. Therefore, a total of 80 hemp fibre substrate blocks were evaluated in this trial. The Rapid Rooter™ grow plugs are in a pre-soaked condition, so the soaking step was eliminated for this treatment. The seeds were of an heirloom bush-type Beefsteak tomato sourced from McKenzie Seeds of Brandon, MB, Canada. One seed was placed in each of the substrate blocks. The prototype hemp fibre substrates and the Jiffy® seed pellets did not have starting holes for seed placement, so tweezers were used to lightly pry open a seed hole, and vermiculite was lightly sprinkled to cover each seed in each sample. All samples were misted 4 times a day during the first two weeks of the trial and reduced to two times a day as plants emerged and established. No fertilizer solutions were applied during the duration of this trial.

Time to germination, root protrusion, and development of true leaves were monitored throughout the 24-day period of the trial.

To generate a baseline of germination for this seed type, a standard petri dish germination trial was run on 3 separate occasions following established rules for testing seeds published in 1987 by the Association of Official Seed Analysts.

Results and Discussion—During the study, it was discovered that Rapid Rooter™ substrate is pre-moistened in a nutrient solution, therefore it was decided to remove it from subsequent discussion as the seedlings were provided with some nutrient solution while all the other treatments were treated only with water.

The overall germination rates promoted by all substrates were found to be not statistically different from each other (Table 1) when total germination was evaluated (Table 2). The highest recruitment rate was with the ROCKWOOL® treatment (91.25%), which is slightly higher than the recruitment rate from the 3 rounds of controlled testing of 90%.

TABLE 1
Analysis of Variance of total Germination Rate

| | | Sum of | Mean | | |
|---|---|---|---|---|---|
| Source | DF | Squares | Square | F Ratio | Prob > F |
| Name | 6 | 153.3571 | 25.5595 | 2.0798 | 0.1069* |
| Rep | 3 | 42.28571 | 14.0952 | 1.1469 | 0.3571 |
| Error | 18 | 221.2143 | 12.2897 | | |
| C. Total | 27 | 416.8571 | | | |

*This shows that there are no statistical differences among all substrates tested.

TABLE 2
Summary Statistics for the Mean Germination Rate
of all Substrates Tested Over a 15-day Time Frame

| | | Mean | | | |
|---|---|---|---|---|---|
| | | Germination | Std | Lower | Upper |
| Substrate | Number | Rate (#/20) | Error* | 95% | 95% |
| 3.5 F | 4 | 15.75 | 1.7528 | 12.067 | 19.433 |
| 3.5 P | 4 | 17 | 1.7528 | 13.317 | 20.683 |
| 5.0 F | 4 | 15.5 | 1.7528 | 11.817 | 19.183 |
| 5.0 P | 4 | 11.5 | 1.7528 | 7.817 | 15.183 |
| Jiffy ® seed | 4 | 18 | 1.7528 | 14.317 | 21.683 |
| pellets | | | | | |
| Rapid Rooter ™ | 4 | 19 | 1.7528 | 15.317 | 22.683 |
| grow plugs | | | | | |
| ROCKWOOL ® | 4 | 18.25 | 1.7528 | 14.567 | 21.933 |

*Std Error uses a pooled estimate of error variance.

The germination over time data shows that the hemp fibre-based substrates had slightly slower germination when compared to the Jiffy® seed pellets and ROCKWOOL®. On average, the seeds in the hemp fibre-based substrates germinated 1-2 days later than the commercial substrates (Table 3). This was not surprising as the hemp fibre substrate blocks did not have the seed placement hole and the seed was therefore more likely to be located closer to the surface of the cube and did not remain as moist as the seeds placed in the ROCKWOOL® and Jiffy® seed pellets. Among the hemp fibre cubes, the two 3.5 formulations had the earliest start, and the highest total germination (see Tables 2 and 3). Further work on the molding of the cubes with a seed placement slot would be likely to improve the germination rate by the one day that it was delayed. ROCKWOOL® is the current standard in large-scale production systems, and these data suggest that the hemp-based substrates: 3.5 F and 3.5 P would be viable alternatives with respect to germination rate.

TABLE 3
Average Time to Germination Measured in Days After Planting

| Days After | | | | | Jiffy ® Seed | |
|---|---|---|---|---|---|---|
| Planting | 3.5 F | 3.5 P | 5.0 F | 5.0 P | Pellets | ROCKWOOL ® |
| 4 | 0 | 0 | 0 | 0 | 1.25 | 0 |
| 5 | 0.5 | 0.25 | 0 | 0 | 8 | 10.25 |
| 6 | 3 | 4.75 | 2.75 | 0.75 | 4.75 | 3 |
| 7 | 3.75 | 4 | 2.25 | 1.75 | 2 | 3.25 |
| 8 | 6 | 4.25 | 7 | 3 | 1 | 1 |
| 9 | 1 | 0.75 | 1.5 | 3 | 0 | 0.5 |
| 10 | 1.25 | 1.25 | 0.75 | 2 | 1 | 0 |
| 11 | 0.25 | 1.25 | 1 | 0.5 | 0 | 0 |
| 12 | 0 | 0 | 0 | 0 | 0 | 0 |
| 13 | 0 | 0.25 | 0.25 | 0.5 | 0 | 0.25 |
| 14 | 0 | 0.25 | 0 | 0 | 0 | 0 |

The time required for the first roots to appear penetrating the outside of the growing substrate was evaluated by visual inspection daily. When a root was observed outside of the media cube it was noted on that day. In general, the roots typically protrude out of the substrate 2-3 days after the seed had germinated. Some of the hemp substrates appeared to have slightly faster root penetration, and this may be due to the rough-cut edges of the substrate cubes, or it may be due to the observation that the hemp substrates have greater number of observable pore spaces which allow the roots to travel throughout the substrate more easily. The data acquired for root protrusion rates are listed in Table 4.

TABLE 4
Average Time to Root Protrusion Through Substrate

| Days After | | | | | Jiffy ® Seed | |
|---|---|---|---|---|---|---|
| Planting | 3.5 F | 3.5 P | 5.0 F | 5.0 P | Pellets | ROCKWOOL ® |
| 6 | 0 | 0 | 0 | 0 | 4.25 | 1 |
| 7 | 0.25 | 1.25 | 0.5 | 0.5 | 5 | 4.25 |
| 8 | 1.75 | 2.25 | 0.75 | 0 | 5 | 3.75 |
| 9 | 4 | 5.25 | 2.25 | 1.5 | 2 | 2.75 |
| 10 | 4.5 | 2.75 | 4.5 | 2.75 | 0.5 | 1.75 |
| 11 | 2.25 | 1.5 | 2 | 1 | 0.5 | 2 |
| 12 | 0 | 1.5 | 0.5 | 0 | 0.25 | 0.5 |
| 13 | 0.25 | 0 | 0 | 0 | 0 | 0 |
| 14 | 0.75 | 1 | 1.75 | 2.5 | 0.5 | 1.5 |
| 15 | 0.5 | 0 | 1 | 0.5 | 0 | 0 |
| 16 | 0.5 | 0 | 0 | 0.25 | 0 | 0 |
| 17 | 0.25 | 0 | 0.25 | 0 | 0 | 0 |
| 18 | 0 | 0 | 0.5 | 0.5 | 0 | 0.25 |
| 19 | 0 | 0 | 0 | 0 | 0 | 0 |
| 20 | 0 | 0.25 | 0.25 | 0 | 0 | 0 |
| 21 | 0.25 | 0.5 | 0.25 | 0.25 | 0 | 0 |

The true leaves were measured on 14 and 20 days after planting. The leaf counts are shown in Table 5.

TABLE 5
Average Leaf Counts 14 and 20 Days After Planting

| | Leaf Count 14 Days | Leaf Count 20 |
|---|---|---|
| Substrate | After Planting | Days After Planting |
| 3.5 F | 2.75 | 5.5 |
| 3.5 P | 3 | 9.5 |
| 5.0 F | 1.75 | 3.25 |
| 5.0 P | 0 | 3.25 |
| Jiffy ® Seed | 27.5 | 36.25 |
| Pellets | | |
| ROCKWOOL ® | 15 | 19.5 |

In general, the commercial substrates performed slightly better with respect to leaf development. This is likely due to two primary reasons (i) Jiffy® seed pellets and ROCKWOOL® likely had better germination performance due to placement of seeds into seed slots in these substrates; the last seedlings emerged on days 8 and 9 after planting. This provides more time for growth and development of true leaves; and (ii) Jiffy® seed pellets have fertilizer and lime which likely contribute to increased growth rates. In addition, the ROCKWOOL® samples were observed to have algae growing therein by the day 10 after planting. The algae could act as a fertilizer source, providing nutrients to the plant.

This initial study of hemp fibre prototypes has indicated that minor improvements in the manufacturing will likely improve the performance of hemp fibre substrates as plant growth substrates. One observation was a lack of consistency of the hemp fibre substrate cubes. The cubes tested in this trial were hand cut and were therefore not identical. As a result, not all cubes were observed to fit tightly into the seed starter trays used. This may have resulted in unnecessary evaporation which may have accounted for the slower germination rate. Lack of a seed placement hole is estimated to cause a two-fold compounding factor that may have accounted for some of the decrease in germination rate, and thus delayed plant development.

The hemp fibre substrate blocks were not pre-soaked in a nutrient solution. This may also be a factor in the delayed rate of germination and leaf development. The decision was made not to use a nutrient solution as most seeds do not need a nutrient solution until 2-7 days after emergence. The plan was to start with a nutrient solution once all seeds had germinated. In the short duration of this trial the decision to not fertilize at all was made due to the slow rate of germination, particularly in the 5.0P formulation which did not see any germination until 8 days after planting in replications 1-3, while replication 4 did see some germination 6 and 7 days after planting. One observation about 5.0P was that it appeared to be more difficult to keep the surface moist as the polymer used in the manufacturing was quite thick on the surface making it stiff and more difficult for water to penetrate. This may be one reason why the 5.0P substrate did not perform as well in this trial. Fertilizer was added to all treatments 22 days after planting, and then weekly until the plants were picked up by their owners. The fertilizer did help to improve leaf development and most of the seedlings successfully turned into viable plants for this year's growing season. This suggests that a second round of trials with the more successful hemp substrates should be performed using a nutrient solution at time of soaking followed by additional nutrient solutions added as true leaves begin to emerge would help in the development of best method practices for this product line.

Black mold was observed developing in some of the hemp fibre blocks. Upon microscopic observation, it was not identifiable as a potential pathogenic mold but rather a mold species that is most likely prolific in the atmosphere. This mold was not observed in the ROCKWOOL® treatments or the other two commercial standards. It is known that black molds can be found in hemp fibres that are used in the automobile industry, particularly when the fibres are processed in a humid environment. One study examined examples of hemp fibres that were inoculated with mold and those that were not inoculated. The mold appeared in both treatments, however was much lower in the case of the non-inoculated treatments. They found in hemp fibres particularly that this recruitment rate was much lower when compared to flax fibres, but it is of some concern for this industry as it may reduce lifespan of the fibre panels in the automotive industry. This insight has led to the recommendation that the oven heating process step be conducted for between about 30 to about 40 minutes or between about 30 to about 60 minutes to ensure that any spores present will be killed during the manufacturing process. Interestingly we did observe some fungal mats on the surface of the potting soil after the cubes were planted into trays for personal use. This fungal mycelium was similar morphologically to the fungi mycelium that develops during the aging process of vermicompost for compost tea production. It is currently believed that white/grey fungal mating is beneficial for the soil biome to help improve plant growth.

This trial demonstrated that the hemp-based substrates for seed starting are a viable alternative to the non-compostable energy intensive ROCKWOOL® and peat-based substrates. It is reasonably predicted that the hemp-based substrates described herein will also be useful for supporting growth of plant clones and as such, uses of the hem-based substrates also encompass growth of clones.

While there were very little statistical differences between the growth parameters examined, the most effective hemp fibre substrate was the 3.5P formulation followed by the 3.5F and 5.0F formulations. There were some issues with the 5.0P formulation that have been previously discussed that may account for its lower overall germination rate and lack of true leaves development.

### Example 2: Application of Amphipathic Glycosides to Hemp Fibres to Improve the Properties of the Hemp Fibre Substrate Blocks

As noted hereinabove, it was recognized that processing of hemp fibres to include a natural biodegradable wetting agent provides the resulting hemp fibre substrate blocks with favorable soaking properties. In the present example, yucca powder which is a source of saponins (amphipathic glycosides), was mechanically mixed with warm or hot water in a 5 gallon pail to a level of 20% concentration, for example 1200 g of yucca extract in 6 L of water. This 20% yucca extract powder solution was then atomized and applied to the hemp fibres in a blow line immediately after the fibres were released from bales. The hemp fibres were carried in an air suspension to a bin through the blow line (a metal tube). The 20% yucca extract solution is added in the metal tube. Application nozzles were used to spray solution in a fan pattern that covers the entire cross section of the blow line. In this process, as the hemp fibres pass through this fan pattern they are coated with the 20% yucca powder solution. It was found that further mixing occurs as the hemp fibres move through the blow line and are discharged into the fibre bin. The target application of yucca extract to hemp fibre is to provide about 2 g of yucca powder to about 310 g of hemp fibre. A typical useful range is between about 1.5 g to 2.5 g of yucca powder to 310 g of hemp fibre. It was determined that this treatment with yucca extract results in individual 1.5″ substrate cubes being completely soaked within 10 minutes after being placed in water, in contrast to 30 minutes for the same sized substrate cubes which are not subjected to the yucca extract treatment, indicating improved ability to soak up water.

### Alternative Embodiments

Various alternative embodiments are within the scope of the invention. While Example 1 describes hemp fibre substrates having about 80% hemp fibre and about 20% biodegradable thermoplastic polymer, it is reasonably predicted that hemp fibre substrates with compositions ranging from between about 70% to about 95% and any integer or fractional value therebetween of hemp fibre and about 5% to about 30% and any integer or fractional value therebetween of at least one biodegradable thermoplastic polymer will provide the substrates with sufficient tensile strength for manipulation during various phases of the manufacturing process and in context of its intended use in growing seeds and clones as well as similar characteristics in terms of aeration, and moisture retention.

While Example 1 describes one type of hemp fibre substrate blocks with dimensions of about 35 mm×about 35 mm×about 35 mm, the hemp fibre substrate may be formed in additional shapes and dimensions such as any shapes and dimensions similar to those described for plant starters and other substrates on grodan101.com (GRODAN Company, ROCKWOOL International A/S, Hovedgaden, Denmark, incorporated herein by reference in its entirety), examples including, but not limited to plant starters in the form of 0.5″ cubic, 1″×1″×1.5″, 1.5″ cubic, or 2″×2″×1.5″ blocks; starter plugs of 2″ sq×H 1.57″; and round plugs with a diameter of 1.5″ and having a slit formed therein for insertion of a cutting or bare root plant; an 8″×8″×8″ block for growing larger plants and small trees; medium sized blocks for transplant of plants with larger roots including blocks with dimensions of 3″×3″×2½″, 3″×3″×4″; 3″×3″×2½″, 4″×4″×3.1″, and 4″×4″×4″; a larger block having dimensions of 6″×6″×6″ for growing plants up to 3-4 feet tall; growing slabs with dimensions of 9.5″×8″×4″ for growing herbs and small plants; and elongated slabs having dimensions of 6″×3″×36″, 8″×3″×36″, 6″×4″×36″, 12″×3″×36″, and 8″×4″×9.5″ for growing large vine crops.

### EQUIVALENTS AND SCOPE

Other than described herein, or unless otherwise expressly specified, all of the numerical ranges, amounts, values and percentages, such as those for amounts of materials, elemental contents, times and temperatures, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Any patent, publication, internet site, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Unless otherwise defined, all technical and scientific terms used herein have the same meaning as commonly understood by one of ordinary skill in the art to which this invention belongs.

While this invention has been particularly shown and described with references to example embodiments thereof, it will be understood by those skilled in the art that various changes in form and details may be made therein without departing from the scope of the invention encompassed by the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context.

It is also noted that the term “comprising” is intended to be open and permits but does not require the inclusion of additional elements or steps. When the term “comprising” is used herein, the term “consisting of” is thus also encompassed and disclosed. Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. Where the term “about” is used, it is understood to reflect +/−10% of the recited value. In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein.

1. A substrate for supporting growth of a seed or a clone, the substrate comprising:
about 70% to about 95% (m/m) hemp fibre and about 5% to about 30% (m/m) of at least one biodegradable thermoplastic polymer.

2. The substrate of claim 1, comprising about 75% to about 85% (m/m) of the hemp fibre and about 15% to about 25% (m/m) of the at least one biodegradable thermoplastic polymer.

3. The substrate of claim 1, comprising about 80% (m/m) of the hemp fibre, and about 20% (m/m) of the at least one biodegradable thermoplastic polymer

4. The substrate of claim 1, wherein the hemp fibre is coated with about 0.3% to about 1.0% (m/m) of a wetting agent.

5. The substrate of claim 4, wherein the wetting agent is a plant extract containing an amphipathic glycoside.

6. The substrate of claim 5, wherein the plant extract is yucca extract.

7. The substrate of claim 1, wherein the hemp fibre has an average size of about 1 mm to about 50 mm.

8. The substrate of claim 1, wherein the hemp fibre is obtained from whole-stalk processing of hemp plants.

9. The substrate of claim 1, wherein the hemp fibre includes bast fibre and hurd fibre.

10. The substrate of claim 1, wherein the biodegradable thermoplastic polymer is in a fibre form.

11. The substrate of claim 1, wherein the biodegradable thermoplastic polymer is compostable.

12. The substrate of claim 1, wherein the biodegradable thermoplastic polymer is polylactic acid, a polyhydroxyalkanoate, a polyesteramide, a polycaprolactone, thermoplastic starch, or poly(butylene adipate co-terephthalate) or a derivative or formulation thereof, wherein the formulation comprises one or more stabilizing additives.

13. The substrate of claim 12, wherein the biodegradable thermoplastic polymer is polylactic acid, or a derivative or formulation thereof, wherein the formulation comprises one or more stabilizing additives.

14. The substrate of claim 1, having a material basis weight between about 2.0 kg/m² to about 6.5 kg/m².

15. The substrate of claim 14, having a material basis weight between about 3.5 kg/m² to about 6.0 kg/m².

16. The substrate of claim 1, having a material basis weight of about 3.5 kg/m².

17. The substrate of claim 1, wherein the hemp fibre and the biodegradable thermoplastic polymer are needle punched to create fibre entanglements therebetween in a fibre air-lay blending process.

18. The substrate of claim 1, in the form of a block having dimensions of about 30 mm×about 30 mm×about 30 mm to about 40 mm×about 40 mm×about 40 mm and having a hole formed substantially centrally on one side for placement of the seed or the clone.

19-35. (canceled)
